(19) United States
(12) Patent Application Publication
(10) Pub. No.: US 2022/0053786 A1
(43) Pub. Date: Feb. 24, 2022

(54) METHODS FOR PRODUCING STIRRED YOGURT

(71) Applicants: AJINOMOTO CO., INC., Tokyo (JP); AMANO ENZYME INC., Nagoya-shi (JP)

(72) Inventor: Tomohiro KODERA, Itasca, IL (US)

(73) Assignees: AJINOMOTO CO., INC., Tokyo (JP); AMANO ENZYME INC., Nagoya-shi (JP)

(21) Appl. No.: 16/997,279

(22) Filed: Aug. 19, 2020

(51) Int. Cl. A23C 9/12; A23C 9/13; A23C 9/127

(52) U.S. Cl. CPC A23C 9/1216; A23C 9/1238; A23C 9/1275; A23C 9/1322

(57) ABSTRACT

A method for producing stirred yogurt, whereby raw milk is treated with a protein glutaminase to form a modified milk prior to fermentation with a starter culture. After fermentation, a gel structure of the resulting yogurt is then disrupted to form the stirred yogurt. Under such a sequence, stirred yogurts are formed having sufficiently high viscosities without the need for thickeners or cross-linking enzymes.

### BACKGROUND OF THE INVENTION

### Field of the Invention

The present invention relates to methods of producing stirred yogurt with protein glutaminase, and stirred yogurts produced by the methods.

### Discussion of the Background

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Enzymes are utilized in various fields in the food industry. Some of the merits of using enzymes in food products include the fact that enzymes act only on specified materials, under mild conditions, and typically do not appreciably influence the taste of the food product.

One enzyme which has attracted recent attention for the modification of dairy products such as yogurt is protein glutaminase (“PG”). PG is a catalytic enzyme that facilitates a deamidation reaction of glutamine residues in proteins by converting the glutamine residue into glutamic acid (amide→carboxylate), thereby increasing the negative charge, the electrostatic repulsion, the hydrating power, and decreasing the isoelectric point of the protein. It is known that, as a result, improvement of various functional characteristics of the protein can be obtained, such as increased solubility and dispersibility. Thus, dairy products treated with PG have been shown to possess a dense and creamy texture that consumers find enjoyable.

For example, U.S. Pat. No. 7,947,315B2—which is incorporated herein by reference in its entirety—describes the production of set yogurts with a smooth oral sensation, decreased hardness, and a suppressed acidic taste by treating raw milk with protein glutaminase.

Similarly, U.S. Pat. No. 8,318,223B2—which is incorporated herein by reference in its entirety—describes set yogurts produced by modifying raw milk materials with protein glutaminase while at the same time improving the effects of the PG by removing natural PG inhibitors contained in the raw milk materials. The set yogurts were described as having a soft and creamy texture.

Set yogurts have a gel-like semi-solid structure where viscosity is generally not a concern—in fact in many cases the viscosity of set yogurts is too high to be mechanically measured. Stirred yogurts (also referred to as “Swiss-style” yogurts), on the other hand, are fluid yogurt products formed as a result of breaking the gel structure at the end of the fermentation (incubation) period, and represent a completely different yogurt product class. Many consumers prefer the lighter, creamier consistency of stirred yogurts, as well as the varieties of stirred fruit and fruit puree options available in stirred yogurt products. However, unlike set yogurts which are not sensitive to changes in viscosity, changes to the viscosity of stirred yogurts is of great concern, and care needs to be taken to ensure acceptable viscosity.

To date, efforts to produce stirred yogurts having sufficiently high viscosity through protein modification with protein glutaminase by itself have not been successful. For example, US 2011/0064847A1—which is incorporated herein by reference in its entirety—describes producing stirred yogurt by adding PG to a raw milk material during the fermentation step (at the same time as a lactic acid bacterium starter culture), and then filtering. The addition of PG during fermentation was found to provide sloppy and soft textured stirred yogurts with no viscosity improvements over control (in which no PG was added).

In another example, US 2019/0021353A1—which is incorporated herein by reference in its entirety—describes producing low-fat stirred yogurt by adding protein glutaminase to nonfat milk during the fermentation step (at the same time as a bacterium starter culture), and then filtering. The resulting stirred yogurt was categorized as ‘unpreferable’ and PG addition was found to substantially lower the viscosity.

Therefore, in attempts to improve the rheological properties (e.g., viscosity) of stirred yogurts, researchers have turned to other additives such as cross-linking enzymes (e.g., transglutaminase, “TG”)(see US 2011/0064847A1—which is incorporated herein by reference in its entirety) or thickeners (e.g., starch)(see US 2019/0021353A1—which is incorporated herein by reference in its entirety) to be added alongside PG (and a starter culture) during the fermentation step. Even when such additive measures have been taken, control of viscosity has still proven difficult. For example, US 2011/0064847A1 reports that modification of raw milk with a combination of PG and TG added together along with a lactic acid bacterium starter culture during fermentation results in decreasing viscosity with escalating dosages of PG. Further, US 2019/0021353A1 discloses that in many cases, the combination of PG and starch provides no improvement or actually reduces viscosity compared to yogurts prepared with only starch thickeners.

### SUMMARY OF THE INVENTION

In view of the forgoing, there is a need for methods of producing stirred yogurts of sufficiently high viscosity, without relying on cross-linking enzymes or thickeners, while also maintaining the familiar dense and creamy texture provided by the action of protein glutaminase.

Accordingly, it is one object of the present invention to provide novel methods for producing stirred yogurts.

It is another object of the present disclosure to provide novel stirred yogurts produced by the inventive methods.

These and other objects, which will become apparent during the following detailed description, have been achieved by the inventors' discovery that modifying milk proteins in raw milk with protein glutaminase prior to carrying out fermentation produces stirred yogurts with excellent rheological properties (e.g., high viscosity and torque) and desirable organoleptic properties (e.g., smoothness, firmness, suppressed acidic taste, and low levels of syneresis).

Thus, the present invention provides:

(1) A method for producing a stirred yogurt, comprising:

treating raw milk with a protein glutaminase to form a modified milk;

fermenting the modified milk in the presence of a starter culture to form a yogurt; and

disrupting a gel structure of the yogurt to form the stirred yogurt.

(2) The method of (1), wherein the raw milk has a fat content of up to 10 wt. %, based on a total weight of the raw milk.

(3) The method of (1) or (2), wherein the raw milk has a protein content of 3 to 10 wt. %, based on a total weight of the raw milk.

(4) The method of any one of (1) to (3), wherein the raw milk is treated with 10 to 500 ppm of the protein glutaminase, based on a total weight of the raw milk.

(5) The method of any one of (1) to (4), wherein the raw milk is treated with the protein glutaminase in an amount of 0.01 to 5 U per gram of protein in the raw milk.

(6) The method of any one of (1) to (5), wherein the raw milk is treated with the protein glutaminase at a treatment temperature of up to 50° C.

(7) The method of any one of (1) to (6), wherein the raw milk is treated with the protein glutaminase for a duration of 15 minutes to 24 hours.

(8) The method of any one of (1) to (7), further comprising heat sterilizing the modified milk after treating the raw milk with the protein glutaminase, and prior to fermenting the modified milk.

(9) The method of (8), wherein the heat sterilizing is performed at a temperature 70 to 95° C. for 1 to 30 minutes.

(10) The method of any one of (1) to (9), further comprising adding a stabilizer to the raw milk prior to treating the raw milk with the protein glutaminase.

(11) The method of any one of (1) to (10), further comprising treating the raw milk with a reducing agent prior to, or concurrently with, the treating of the raw milk with the protein glutaminase.

(12) The method of (11), wherein the raw milk is treated concurrently with the reducing agent and the protein glutaminase.

(13) The method of (11) or (12), wherein the raw milk is treated with 10 to 500 ppm of the reducing agent, based on a total weight of the raw milk.

(14) The method of any one of (11) to (13), wherein the reducing agent is yeast extract.

(15) The method of any one of (1) to (14), wherein the modified milk is fermented with 0.0001 to 1 wt. % of the starter culture, based on a total weight of the modified milk.

(16) The method of any one of (1) to (15), which does not involve the use of a thickener.

(17) The method of any one of (1) to (16), further comprising treating the raw milk with a transglutaminase enzyme concurrently with the treating of the raw milk with the protein glutaminase.

(18) The method of any one of (1) to (16), wherein the protein glutaminase is the only enzyme utilized that acts on glutamine residues of milk proteins contained in the raw milk.

(19) A stirred yogurt produced by the method of any one of (1) to (18).

### BRIEF DESCRIPTION OF THE DRAWINGS

The foregoing paragraphs have been provided by way of general introduction, and are not intended to limit the scope of the following claims. The described embodiments, together with further advantages, will be best understood by reference to the following detailed description when considered in conjunction with the accompanying drawings, wherein:

FIGS. 1A-1B are graphs showing the viscosity (FIG. 1A) and torque (FIG. 1B) of stirred yogurt samples #1 and 2 after 7 days cold storage;

FIGS. 2A-2B are images of stirred yogurt samples #1 (FIG. 2A) and #2 (FIG. 2B) after 7 days of cold storage;

FIGS. 3A-3B are graphs showing the viscosity (FIG. 3A) and torque (FIG. 3B) of stirred yogurt samples #3-9 after 10 days of cold storage;

FIGS. 4A-4G are images of stirred yogurt samples #3 (FIG. 4A), #4 (FIG. 4B), #5 (FIG. 4C), #6 (FIG. 4D), #7 (FIG. 4E), #8 (FIG. 4F), and #9 (FIG. 4G) after 10 days of cold storage;

FIGS. 5A-5C are graphs showing the viscosity of stirred yogurt samples #10-17 after 1 and 25 days of cold storage;

FIGS. 6A-6C are graphs showing the torque of stirred yogurt samples #10-17 after 1 and 25 days of cold storage.

### DETAILED DESCRIPTION OF THE INVENTION

In the following description, it is understood that other embodiments may be utilized and structural and operational changes may be made without departure from the scope of the present embodiments disclosed herein.

### Definitions

As used herein, the phrase “substantially free”, unless otherwise specified, refers to a composition/material which contains less than 1 wt. %, preferably less than 0.5 wt. %, preferably less than 0.3 wt. %, preferably less than 0.2 wt. %, preferably less than 0.1 wt. %, preferably less than 0.05 wt. %, preferably less than 0.03 wt. %, preferably less than 0.02 wt. %, preferably less than 0.01 wt. %, preferably less than 0.001 wt. %, preferably less than 0.0001 wt. %, preferably 0 wt. % of a particular component, relative to a total weight of the composition/material.

As used herein, the terms “optional” or “optionally” means that the subsequently described event(s) can or cannot occur or the subsequently described component(s) may or may not be present (e.g., 0 wt. %).

The term “raw milk” is broadly used in the present application to refer to a composition based on milk or milk components obtained from mammals which has not been subject to enzymatic treatment for alteration of proteins contained therein, and which can be used as a medium for growth and fermentation of lactic acid bacteria. Included are those compositions which have been defatted, supplemented, pasteurized, homogenized, fortified, concentrated, diluted, or otherwise processed. Conversely, the term “modified milk” herein refers to those compositions based on milk or milk components which have been subject to enzymatic treatment for alteration of proteins contained therein.

As used herein a “set yogurt” is a non-fluid yogurt product obtained after precipitation of milk proteins. Set yogurts are formed by leaving the milk product undisturbed throughout the fermentation (incubation) and cooling period, which provides the set yogurt with a continuous gel-like (semi-solid) structure.

On the other hand, a “stirred yogurt” is a fluid yogurt product formed as a result of breaking the gel structure at the end of the fermentation (incubation) period and prior to cooling and further processing. Stirred yogurts thus have different textural and rheological properties (e.g., lower viscosities) from set yogurts. Stirred yogurts having sufficiently high viscosities can be handled practically with a spoon (“spoonable”), and are especially appreciated.

The term “short texture” refers to the opposite of ropy texture. So when the ropyness of a yogurt decreases, the texture becomes “shorter”. A “ropy” texture refers to a texture characteristic of the yogurt assessed by sensory evaluation where a spoon spoonful of yogurt is pulled from the sample and the ropy property is evaluated; the longer the thread becomes before it breaks the ropier the product.

### Methods for Producing Stirred Yogurts

The present disclosure is directed to methods of producing stirred yogurts having excellent rheological properties (e.g., high viscosity) and agreeable organoleptic properties (e.g., smoothness, firmness, mild acidic taste, low syneresis), by modifying milk proteins in raw milk with protein glutaminase prior to carrying out fermentation.

Thus, the methods of the present disclosure generally involve (at least) the following sequence:

(i) treating raw milk with a protein glutaminase to form a modified milk; then

(ii) fermenting the modified milk by adding a starter culture to form a yogurt; and then
(iii) disrupting a gel structure of the yogurt to form the stirred yogurt.

### Raw Milk

The raw milk to be modified with protein glutaminase in the present disclosure is not particularly limited, and may be any edible milk, for example, milk obtained from a cow, a buffalo, a goat, a sheep, a horse, a camel, a yak, etc. For each of the forgoing, a pasteurized milk (e.g., a low temperature and long sterilization time milk—“LTLT milk”, a high temperature and short sterilization time milk—“HTST milk”, an ultra-high temperature heating sterilization milk—“UHT milk”), a milk formulated in components such as protein and/or milk fat, a fortified milk containing additional vitamins and minerals, a defatted milk, a homogenized milk, a processed milk, a diluted milk, a concentrated milk, a dried milk (powdered milk), a defatted dry milk, a defatted milk solution, a dried (powdered) milk suspended and dissolved in water, a lactose-reduced milk, etc. are included in this category.

Raw milk having a wide range of fat contents may be utilized herein. Typically, the raw milk has a fat content of up to 10 wt. %, preferably up to 8 wt. %, preferably up to 6 wt. %, preferably up to 5 wt. %, preferably up to 4 wt. %, preferably up to 3 wt. %, preferably up to 2 wt. %, preferably up to 1.5 wt. %, preferably up to 1 wt. %, preferably up to 0.5 wt. %, preferably up to 0.3 wt. %, preferably up to 0.1 wt. %, preferably 0 wt. %, based on a total weight of the raw milk. Specific mention is made to commercially available milk products such as whole milk (fat content of about 3.25 wt. %), reduced fat milk (fat content of about 2 wt. %), low fat milk (fat content of about 1 wt. %), and skim milk (fat content of about 0 to 0.5 wt. %). In some embodiments, it may be desirable to use raw milk which has been altered in terms of fat content, for example low fat milk or skim milk, for preparing low fat stirred yogurt products.

The raw milk may also have a wide range of protein contents, with typical protein content values being at least 0.5 wt. %, preferably at least 1 wt. %, preferably at least 1.5 wt. %, preferably at least 2 wt. %, preferably at least 2.5 wt. %, preferably at least 3 wt. %, and up to 10 wt. %, preferably up to 8 wt. %, preferably up to 6 wt. %, preferably up to 5.5 wt. %, preferably up to 5 wt. %, preferably up to 4.5 wt. %, preferably up to 4 wt. %, preferably up to 3.5 wt. %, based on a total weight of the raw milk. In some embodiments, it may be desirable to use raw milk which has been altered in terms of protein content, for example protein supplemented raw milk, for preparing high protein content stirred yogurt products.

While raw milk having fat and protein contents outside of the above-mentioned ranges may be utilized in some circumstances, preferred raw milks are those commercial raw milks or raw milks that have been adjusted to fall within the above-mentioned ranges according to methods known to those of ordinary skill in the art.

### Stabilizer

The methods of the present disclosure may optionally involve addition of a stabilizer, for example, to help prevent syneresis. The stabilizer may be optionally added during any stage of stirred yogurt production. For example, the stabilizer may be added to raw milk, that is, prior to treating the raw milk with the protein glutaminase; to modified milk, that is, after treatment with PG and prior to fermentation; to yogurt, that is, after fermentation; or the stabilizer may be added during two or more of these stages. Addition of stabilizer to raw milk prior to treating the raw milk with protein glutaminase is especially preferred.

When employed, the stabilizer may be added in an amount of at least 0.01 wt. %, preferably at least 0.05 wt. %, preferably at least 0.1 wt. %, preferably at least 0.2 wt. %, preferably at least 0.4 wt. %, preferably at least 0.6 wt. %, preferably at least 0.8 wt. %, preferably at least 1 wt. %, and up to 5 wt. %, preferably up to 4.5 wt. %, preferably up to 4 wt. %, preferably up to 3.5 wt. %, preferably up to 3 wt. %, preferably up to 2.5 wt. %, preferably up to 2 wt. %, preferably up to 1.5 wt. %, based on a total weight of the composition to which it is added, for example when added to raw milk, based on a total weight of the raw milk.

Examples of stabilizers which may be utilized herein include, but are not limited to, pectin, agar, carrageenan, gelatin, whey protein concentrates, chicory root fiber, and modified food starch, as well as mixtures thereof, with specific mention being made to one or more of pectin, modified food starch, and gelatin, preferably two or more of pectin, modified food starch, and gelatin.

<Pectin> Pectin is a structural heteropolysaccharide, rich in galacturonic acid, contained in the primary cell walls of terrestrial plants. A wide range of pectins are suitable for use as a stabilizer in the present disclosure, in particular, commercial pectins extracted from fruit such as citrus peel or apple peel. While the use of high-methoxyl (HM) pectins (i.e., more than 50% of all the galacturonic acid units are methyl esterified) are contemplated, preferred pectins are those which are categorized as low-methoxyl (LM) pectins (i.e., 50% or less of all the galacturonic acid units are methyl esterified). For example, the pectin may have less than 45%, preferably less than 40%, preferably less than 35%, preferably less than 30%, preferably less than 25%, preferably less than 20%, preferably less than 15%, preferably less than 10%, preferably less than 5% of a total galacturonic acid content in the form of a methyl ester. The non-esterified galacturonic acid units of the pectin used herein can be either free acids (carboxylic acid groups) or salts with sodium, potassium, calcium and/or ammonium ions. Specific examples of pectin stabilizers that may be used herein include, but are not limited to, UNIPECTIN products available from Cargill.

<Modified food starch> Modified food starch is a complex carbohydrate derived from graincereals, vegetables, root vegetables, legumes, and fruits, such as from wheat, rice, corn (maize), potato, taro, yams, squash, beans, cassava, etc. that has had one or more of its components altered physically, chemically, or enzymatically, for example, to provide a more desirable texture, heat and/or acidic resistance, and/or solubility/swellability profile compared to regular starch (unmodified). The modification of starch may be performed by any method known to those of ordinary skill in the art, such as acid treatment, alkaline treatment, bleaching, oxidizing, enzyme treatment, phosphorylation, crosslinking (e.g., with sodium trimetaphosphate), acetylation, hydroxypropylation/hydroxyethylation, carboxymethylation, and the like, as well as mixtures thereof (e.g., crosslinked and stabilized starches such as hydroxypropyl distarch phosphate). Specific examples of modified food starches that may be used herein include, but are not limited to, chemically modified starches derived from waxy maize or tapioca (e.g., THERMTEX and NATIONAL products, each available from Ingredion), and SHUR STAB Cultured Dairy Systems available from Denali.

<Gelatin> Gelatin is a mixture of proteins and peptides which is produced by the partial hydrolysis (breakdown) of collagen extracted from the skin, bones, and/or connective tissues of animals, and various gelatins may be useful as a stabilizer herein, such as gelatin obtained from domesticated cattle, chicken, pigs, and marine sources, in particular, bovine bones and hide. In general, either Type A gelatin (obtained from acid-treated raw material) or Type B gelatin (obtained from alkali-treated raw material) may provide acceptable stabilization to the stirred yogurts, with specific mention being made to Type B gelatin, more preferably Type B bovine (beef) gelatin. The gelatin of the present disclosure may be a very low bloom gelatin (bloom number of <50); a low bloom gelatin (bloom number of 50 to <150); a medium bloom gelatin (bloom number of 150 to <220); or a high bloom gelatin (bloom number of 220 or greater), according to the bloom gel strength test procedure from the Gelatin Manufactures Institute of America (Standard Methods for the Sampling and Testing of Gelatins, Gelatin Manufacturers Institute of America, Inc., 1986, 501 fifth Ave. New York, N.Y., as well as The Association of Analytical Communities (AOAC) international, AOAC method 948.21 “Jelly Strength of Gelatin”—each incorporated herein by reference in its entirety). The bloom number is the force (in grams) required to depress a standard AOAC plunger (12.7 mm diameter flat face cylindrical probe with a sharp edge) 4 mm into a set gelatin of 6.66% (w/v) concentration (e.g., 7.5 g gelatin in 105 mL of water) that has been kept at 10° C. for 16 hours. In preferred embodiments, the gelatin is a high bloom gelatin with a bloom number of at least 220, preferably at least 230, preferably at least 240, preferably at least 250, and up to 325, preferably up to 300, preferably up to 290, preferably up to 280, preferably up to 260.

In some embodiments, mixtures of two or more stabilizers may be employed in the disclosed methods. Exemplary mixtures may include, but are not limited to, a mixture of pectin and modified food starch, a mixture of whey protein concentrates and pectin (e.g., VITEX AYS 08, available from Cargill), and a mixture of agar and pectin (e.g., VITEX AYS 10, available from Cargill). When a mixture of a first stabilizer and a second stabilizer is employed, for example a mixture of pectin and modified food starch, a weight ratio of the first stabilizer to the second stabilizer may be from at least 1:20, preferably at least 1:15, preferably at least 1:10, preferably at least 1:5, preferably at least 1:3, preferably at least 1:2, preferably at least 1:1, and up to 20:1, preferably up to 15:1, preferably up to 10:1, preferably up to 5:1, preferably up to 3:1, preferably up to 2:1.

Protein Glutaminase

The methods of the present disclosure involve treating raw milk, preferably raw milk to which a stabilizer has been added, with a protein glutaminase (PG) to deamidate proteins contained in the raw milk thereby forming a modified milk.

Prior to adding the protein glutaminase, the raw milk may be optionally brought to a temperature at which the protein modification is to be performed (the treatment temperature). For example, the raw milk may be brought to a temperature of up to 60° C., preferably up to 50° C., for example, at least 10° C., preferably at least 15° C., preferably at least 20° C., preferably at least 25° C., preferably at least 30° C., and up to 60° C., preferably up to 55° C., preferably up to 50° C., preferably up to 45° C., preferably up to 40° C., preferably up to 35° C., and this temperature may be maintained for at least 1 minute, preferably at least 5 minutes, preferably at least 10 minutes, preferably at least 15 minutes, preferably at least 20 minutes, and up to 60 minutes, preferably up to 45 minutes, preferably up to 40 minutes, preferably up to 35 minutes, preferably up to 30 minutes, prior to adding the PG. In preferred embodiments, the raw milk is brought to a temperature of about 45 to 50° C. and this temperature is maintained for about 30 to 35 minutes, prior to addition of the PG.

The protein glutaminase is then added to the raw milk, preferably to raw milk already at the treatment temperature for carrying out the deamidation reaction. As the protein glutaminase of the present disclosure, a commercially available PG or a PG prepared from a culture medium of a microorganism producing protein glutaminase can be used—the kind of protein glutaminase used is not particularly limited as long as it directly acts on glutamine residues in protein(s) contained in the raw milk to deamidate the same without peptide bond cleavage and/or crosslinking of the protein(s). Examples of such protein glutaminases include, but are not limited to, protein glutaminases derived from Chryseobacterium, Flavobacterium or Empedobacter, such as those disclosed in JP-A-2000-50887, JP-A-2001-218590, WO 2006/075772—each incorporated herein by reference in its entirety—including commercially available protein glutaminase derived from Chryseobacterium, and the like. A specific example of a protein glutaminase that may be used in the methods of the present disclosure includes, but is not limited to, protein glutaminase (500 U/g, available from Amano Enzyme, Inc.).

The protein glutaminase can be prepared from a culture liquid for microorganism that produces the protein glutaminase, for example, using known methods for protein separation and purification (e.g., centrifugation, ultrafiltration (UF) concentration, salting out, various chromatographies using an ion exchange resin, etc.). For example, a culture medium may be centrifuged to remove bacteria, after which salting out, chromatography, and the like, may be combined to give the object enzymes. When collecting PG enzymes from the interior of bacterial cells, the bacterial cells may be recovered from a culture liquid by filtration or centrifuge, etc., and then the bacterial cells can be crushed by, for example, a pressure processing or ultrasonic processing technique. Protein separation and purification methods such as those described above may then be performed to obtain the target PG enzymes. The enzymes may be powdered by a drying method such as a freeze drying or vacuum drying, etc., with optional use of an appropriate diluent or drying aid.

In some embodiments, the activity of the protein glutaminase may be measured by the following steps:

(1) 100 μL of an aqueous solution containing the protein glutaminase is added to 1 ml of 0.2 M phosphoric acid buffer solution (pH 6.5) containing 30 mM of Z-Gln-Gly (Peptide Institute, Inc.), and incubated for 10 minutes at 37° C., and then the reaction is ceased by adding 1 ml of 0.4 M trichloroacetic acid (TCA) solution.

(2) An amount of ammonia produced by the reaction in a reacted solution of (1) is measured by using Ammonia-test-Wako (manufactured and retailed by Waka Pure Chemical Industries, Ltd.).

(3) A blank solution is measured at the same time. The blank solution is prepared such that a solution without the enzyme is incubated for 10 minutes at 37° C., and then 1 ml of 0.4M TCA solution and 100 μL of an aqueous solution containing the protein glutaminase enzyme are added in this order.

(4) The concentration of the enzyme is adjusted by diluting with a phosphoric acid buffer solution (pH 6.5) such that an absorbance at 630 nm becomes in the range of 40.2 to 0.8.

(5) The activity is measured by a unit (U) which indicates the amount of the enzyme that can produce one μmol of ammonium in one minute. The enzyme activity is calculated by a following expression.

Enzyme activity (U/ml)=(Es−Eb)*F*0.123×Df

where;
Es: absorbance of a solution enzyme reacted,
Eb: absorbance of a blank,
F: factor (the reciprocal of the slope of a straight line of a calibration curve for standard ammonium solution), and
Df: dilution ratio of an enzyme solution.

It is possible to adjust the degree of deamidation of proteins contained in the raw milk, i.e., the degree of modification of raw milk, in order to achieve the desired material characteristics of the stirred yogurts, by controlling the treatment temperature, the treatment duration, the amount of PG utilized, etc.

In terms of the treatment temperature, the raw milk is typically treated with the protein glutaminase at a treatment temperature of up to 60° C., preferably up to 50° C., for example, at least 10° C., preferably at least 15° C., preferably at least 20° C., preferably at least 25° C., preferably at least 30° C., and up to 60° C., preferably up to 55° C., preferably up to 50° C., preferably up to 45° C., preferably up to 40° C., preferably up to 35° C., with 45 to 50° C. being the most preferred.

In terms of the treatment duration, the raw milk may be treated with the protein glutaminase for up to 24 hours. For example, the raw milk may be treated with the protein glutaminase for a duration of at least 15 minutes, preferably at least 30 minutes, preferably at least 45 minutes, preferably at least 50 minutes, preferably at least 55 minutes, preferably at least 60 minutes, and up to 24 hours, preferably up to 12 hours, preferably up to 8 hours, preferably up to 4 hours, preferably up to 2 hours, preferably up to 1.5 hours, with 45 to 60 minutes being the most preferred.

The amount of protein glutaminase to be added can be varied according to the composition of raw milk (e.g., protein content), a kind of protein to be modified, or a desired effect to be obtained. For example, the amount of PG added may be at least 0.01 U per g of protein in the raw milk, preferably at least 0.1 U per g, preferably at least 0.5 U per g, preferably at least 1 U per g, preferably at least 1.5 U per g, preferably at least 2 U per g, preferably at least 2.5 U per g of protein in the raw milk, and up to 10 U per g of protein in the raw milk, preferably up to 8 U per g, preferably up to 6 U per g, preferably up to 5 U per g, preferably up to 4 U per g, preferably up to 3 U per g of protein in the raw milk.

In terms of weight of raw milk, the raw milk may be treated with at least 10 ppm, preferably at least 20 ppm, preferably at least 30 ppm, preferably at least 40 ppm, preferably at least 50 ppm, preferably at least 60 ppm, preferably at least 70 ppm, preferably at least 80 ppm, preferably at least 90 ppm, preferably at least 100 ppm, and up to 500 ppm, preferably up to 450 ppm, preferably up to 400 ppm, preferably up to 350 ppm, preferably up to 300 ppm, preferably up to 250 ppm, preferably up to 200 ppm, preferably up to 150 ppm, preferably up to 125 ppm of the protein glutaminase, based on a total weight of the raw milk. Of course, PG dosages outside of this range may also be used as needed in order to obtain desired levels of deamidation/effects from the protein modification.

While enzymes other than protein glutaminase may be optionally employed for protein modification herein, one benefit of the present disclosure is that the use of other protein modification enzymes is not required in order to produce stirred yogurts of sufficiently high viscosity. At least in terms of lowering operational costs and reducing complexity, it is preferred that protein glutaminase is the only enzyme used for protein modification, e.g., the only enzyme used that acts on glutamine residues of milk proteins contained in raw milk. Another type of enzyme that acts on glutamine residues, which may be used to treat the raw milk concurrently or in conjunction with the PG, but is preferably excluded from the methods of the present disclosure, are crosslinking enzymes such as transglutaminase (TG). Transglutaminase is not a deamidating enzyme because it functionally cross-links proteins via glutamine and amino-containing residues (e.g., lysine) and provides almost no deamidation. Both calcium independent TG types (e.g., TG obtained from microorganisms such as an actinomycete or Bacillus subtilis, etc.) and calcium dependent TG types (e.g., those obtained from the liver of guinea pigs or the blood of cattle or pigs, human epidermal keratin cells, human blood coagulation factor XIII, microorganisms such as oomycete, those obtained from fish and oysters, etc.) may be used in addition to PG, but are preferably excluded in the disclosed methods, for example, those described in US 2011/0064847A1—incorporated herein by reference in its entirety.

Specific examples of transglutaminase enzymes or preparations containing transglutaminase enzymes include, but are not limited to, ACTIVA MP (a transglutaminase enzyme designed for dairy applications), available from Ajinomoto Foods Europe; and ACTIVA YG (a transglutaminase preparation including yeast extract designed for dairy applications), available from Ajinomoto Health & Nutrition North America, Inc Combinations of transglutaminase enzymes and protein glutaminase enzymes are also commercially available, such as ACTIVA SYG (a combination of protein glutaminase and transglutaminase enzymes designed for dairy applications), available from Ajinomoto Foods Europe.

### Reducing Agent

A reducing agent may also be optionally employed in the disclosed methods, for example, to diminish/remove any inhibitors present in the raw milk which may interfere with the protein glutaminase enzyme. Thus, the use of a reducing agent may increase the effectiveness of PG treatment, and in some cases, allow for lower dosages of PG to reach the same level of effectiveness. Other advantages provided by use of reducing agents may include fermentation acceleration, starter replacement, and taste/texture improvement.

The amount of reducing agent to be used may be adjusted, for example, based on the activity of the reducing agent selected, the type of raw milk to be treated, and the desired taste profile of the stirred yogurt. Generally, the reducing agent may be added in an amount of up to 10,000 ppm. A person of ordinary skill in the art will be able to determine the proper amount of reducing agent to be used, and adjust as needed. Most typically, when employed, the reducing agent may be added in an amount of at least 10 ppm, preferably at least 20 ppm, preferably at least 30 ppm, preferably at least 40 ppm, preferably at least 50 ppm, preferably at least 60 ppm, preferably at least 70 ppm, preferably at least 80 ppm, preferably at least 90 ppm, preferably at least 100 ppm, and up to 500 ppm, preferably up to 450 ppm, preferably up to 400 ppm, preferably up to 350 ppm, preferably up to 300 ppm, preferably up to 250 ppm, preferably up to 200 ppm, preferably up to 150 ppm, preferably up to 125 ppm of the reducing agent, based on a total weight of the raw milk.

Suitable reducing agents include, but are not limited to, thiol compounds such as a glutathione, cysteine, γ-glutamilcysteine, yeast extract containing at least one such thiol compound, thiosulfuric acid, sulfurous acid, ascorbic acid, erythorbic acid and salts thereof which are allowed to be used as food additives; and tocopherols; with yeast extract being particularly preferred. With respect to yeast extract, any yeast extract containing glutathione can be applied in the disclosed methods to, among other benefits, increase the texture of the stirred yogurt which has been treated by PG. As a non-limiting example, yeast extract containing a glutathione content of at least 4 wt. %, preferably at least 6 wt. %, preferably at least 8 wt. %, and up to 25 wt. %, preferably up to 20 wt. %, preferably up to 15 wt. %, may be used as reducing agent herein. Suitable examples of which include, but are not limited to, AROMILD U, a yeast extract containing 8 wt. % of natural glutathione, available from Kohjin.

As to the timing of addition, the raw milk may be treated with the reducing agent prior to treatment with protein glutaminase, or concurrently with treatment with protein glutaminase. From a practical standpoint, as well as in terms of maximizing the effect of the reducing agent, it is preferred that the raw milk be treated concurrently with the reducing agent and the protein glutaminase, for example, by adding the reducing agent and the PG to raw milk at the same time, or substantially at the same time (e.g., sequentially), and then subjecting the raw milk to the treatment temperature and treatment duration described above for protein modification with PG.

### Heat Sterilizing

The deamidation reaction may optionally be monitored, for example, by measuring an amount of ammonium produced during the treatment operation with PG. After sufficiently treating the raw milk with the protein glutaminase to form a modified milk, and prior to fermenting the modified milk, the modified milk may be subject to heat sterilization to cease protein modification by deactivating the protein glutaminase enzyme, as well as any other enzymes or live organisms present. This heat treatment may also denature the milk proteins (including the modified milk proteins) so that they do not form curds.

Any sterilization conditions used for manufacturing dairy products may be generally employed herein. In preferred embodiments, the modified milk is heat sterilized at a temperature of at least 70° C., preferably at least 75° C., preferably at least 80° C., preferably at least 85° C., and up to 95° C., preferably up to 90° C. Depending on the temperature employed, the heat sterilizing may be performed for e.g., at least 1 minute, preferably at least 2 minutes, preferably at least 4 minutes, preferably at least 5 minutes, preferably at least 10 minutes, preferably at least 15 minutes, and up to 30 minutes, preferably up to 25 minutes, preferably up to 20 minutes. Of course, it may be possible to obtain the effect of the present disclosure without such a heat sterilization process.

### Fermentation

Fermentation of the modified milk involves the addition of a starter culture to the modified milk, which is a culture of food-grade micro-organisms, in particular thermophilic lactic acid bacteria (e.g., Streptococcus spp. and Lactobacillus spp.), which are responsible for the acidification of the modified milk. During the fermentation stage, the consumption of lactose by these bacteria causes the formation of lactic acid, reducing the pH and leading to the formation of a protein coagulum. This acidification and coagulation of the modified milk thus produces the yogurt.

Specifically, the starter culture used for fermentation herein is Streptococcus thermophilus and Lactobacillus delbrueckii Subsp. Bulgaricus and optionally other microorganisms such as Lactobacillus delbrueckii Subsp. lactis, Bifidobacterium animalis Subsp. lactis, Lactococcus lactis, Lactobacillus acidophilus, and Lactobacillus paracasei, or any microorganism derived therefrom. The lactic acid strains other than Streptococcus thermophilus and Lactobacillus' delbrueckii Subsp. bulgaricus, may optionally be included to give the finished yogurt product various properties, such as the property of promoting the equilibrium of the flora. The starter cultures may be fresh, frozen or freeze dried.

Specific examples of starter cultures that may be used to ferment the modified milk of the present disclosure include, but are not limited to, YOFLEX products such as YC-X11, YC-180 YC-280 YC-370 YC-380, YC-381, and PREMIUM 1.0, as well as NU-TRISH products such as ABT-1, ABT-10, ABY-1, and ABY-10, each available from Chr. Hansen. It should also be mentioned that yogurts containing remaining live lactic acid bacteria may also be used as starter culture for carrying out fermentation.

Typical starter culture amounts range from at least 0.0001 wt. %, preferably at least 0.0005 wt. %, preferably at least 0.001 wt. %, preferably at least 0.005 wt. %, preferably at least 0.01 wt. %, preferably at least 0.015 wt. %, preferably at least 0.02 wt. %, and up to 1 wt. %, preferably up to 0.5 wt. %, preferably up to 0.2 wt. %, preferably up to 0.1 wt. %, preferably up to 0.05 wt. %, preferably up to 0.03 wt. %, based on a total weight of the modified milk.

If the modified milk is at an elevated temperature, such as from a heat sterilizing operation, the modified milk may first be cooled to an appropriate fermentation temperature, prior to addition of the starter culture. Once the starter culture has been added, the fermentation may be carried out at temperature of at least 22° C., preferably at least 26° C., preferably at least 30° C., preferably at least 34° C., preferably at least 38° C., preferably at least 42° C., and up to 45° C., preferably up to 44° C., preferably up to 43° C. The modified milk may be fermented until a pH of at least 4.0, preferably at least 4.2, preferably at least 4.4, and up to 5.0, preferably up to 4.8, preferably up to 4.6 is reached, which generally corresponds to a fermentation time of at least 1 hour, preferably at least 2 hours, preferably at least 3 hours, preferably at least 4 hours, and up to 10 hours, preferably up to 8 hours, preferably up to 6 hours. It should be understood that these conditions can be appropriately modified or adjusted according to the purity of starter culture to be used, the kind and purity of protein in the modified milk, etc.

### Disrupting Gel Structure

To produce stirred yogurt, the gel structure (coagulum) of the yogurt produced after fermentation is next disrupted. The gel structure may be disrupted by any technique known by those of ordinary skill in the art, for example, by stirring/mixing (e.g., kitchen mixer), filtering/sieving (e.g., pore size of 200 to 500 μm), and the like to form the stirred yogurt. In preferred embodiments, the gel structure of the yogurt is disrupted via stirring. The production of stirred yogurt may also involve one or more of pumping, cooling (e.g., 4 to 15° C.), and packaging the stirred yogurt for cold storage and/or distribution/sale.

### Additive

Additives generally used for the production of yogurt can be optionally used in the present methods. Exemplary additives include, but are not limited to, added dairy raw materials (e.g., cream); sugar or sweetening agents (e.g., sucrose, maltitol, sorbitol, lactose); oil and fat; emulsifiers; flavorings including seasonings and spices; colorants; antioxidants; fruits (e.g., fruit pulp and fruit juice of strawberry); cereals; thickeners (e.g., starch); nutritional substances such as vitamins, minerals, and fiber (e.g., vitamin A, vitamin B, vitamin D, calcium, zinc, iron, folic acid, riboflavin, dextrin); animal and/or vegetable protein (e.g., soybean protein, wheat protein, etc.); and solid foods (e.g., chocolate). As to the timing of addition, when employed, the additive(s) may be added at art appropriate addition points. For example, when stirred yogurt products containing fruit are desired, the fruit additive may be added during the step of disrupting the gel structure of yogurt produced from fermentation by stirring the fruit additive into the yogurt during the stirring operation.

Particular mention is made to thickeners. While the use of any suitable food-grade thickener known to those of ordinary skill in the art is contemplated (may be optionally employed), it is preferred that the order of operation specified in the present disclosure is sufficient for providing the stirred yogurts with a desirable viscosity increase, without the need for added thickeners. Thus, in preferred embodiments, no thickeners are added during the manufacture of the stirred yogurts. Examples of thickeners include, but are not limited to, starch such as starch derived from potatoes (e.g., white potato, sweet potato, etc.), graincereals (e.g., wheat, rice, corn, etc.), vegetables, root vegetables, or fruits, including both pregelatinized and unpregelatinized starch, for example starch(es) described in US 2019/0021353A1—which is incorporated herein by reference in its entirety; xanthan gum; guar gum; targacanth; alginates (e.g., sodium alginate, potassium alginate, ammonium alginate, and/or calcium alginate); karaya gum; carob; furcelleran; locust bean gum; tapioca; gum arabic (acacia); modified alginates (e.g., propylene glycol alginates); and modified cellulose polymers such as hydroxypropylmethylcellulose (HPMC), methylcellulose (MC), carboxymethylcellulose (CMC), hydroxymethylcellulose, hydroxypropylcellulose, and microcrystallinecellulose; as well as mixtures thereof. In preferred embodiments, starch is not utilized in the disclosed methods, for example, as a way to increase viscosity.

A particularly preferred production method of stirred yogurt in the present disclosure may include preparing raw milk with a fat and protein content within the above-mentioned ranges, adding one or more stabilizers (e.g., pectin and modified food starch) thereto as necessary, and pre-heating the raw milk to the desired treatment temperature (e.g., 45 to 50° C.). Next, the raw milk may be treated with protein glutaminase in the presence of a reducing agent (e.g., yeast extract) at the selected treatment temperature (e.g., 45 to 50° C.) and for an appropriate treatment duration (e.g., 45 to 60 minutes) to generate a modified milk. Treatment with PG may be stopped by subjecting the modified milk to heat sterilization, for example at a temperature of 80 to 95° C. for 15 to 30 minutes. Then, after heat sterilizing and cooling by a conventional method, a starter culture comprising Streptococcus thermophilus and Lactobacillus delbrueckii Subsp. Bulgaricus may be added and incubated in the modified milk in a tank at 38 to 45° C. to ferment the modified milk and produce yogurt. After fermentation is deemed complete, the yogurt is preferably stirred to disrupt the coagulum and form the stirred yogurt, which may be stored under reduced temperature (e.g., 5 to 10° C.).

As discussed above, the methods of the present disclosure involve treating raw milk with protein glutaminase to form a modified milk prior to fermentation with a starter culture. The inventors have unexpectedly discovered that only when such a process sequence is followed, are stirred yogurts produced having suitable rheological properties (e.g., high viscosity and torque). For example, it has been found that when raw milk is modified with protein glutaminase prior to fermentation, the viscosity of the stirred yogurt increases by up to 60% relative to control (no PG treatment) (e.g., see Example 3, sample #16 compared to control sample #10).

On the other hand, when protein glutaminase is added during the fermentation step, the resulting stirred yogurts have been found to have a lower viscosity compared to control (no PG treatment) (e.g., see Example 2, sample #9 compared to control sample #3), which is also in agreement with previous efforts to modify stirred yogurt products using protein glutaminase.

Thus, the increased viscosities obtained by the disclosed methods are especially unexpected when one considers previous efforts to use PG alone for making stirred yogurts actually reduced the viscosity of the stirred yogurt products (see US 2011/0064847A1 and US 2019/0021353A1—each incorporated herein by reference in its entirety)—which in light of the present disclosure can now be understood to be due to the untimely addition of PG during fermentation.

### Stirred Yogurt

The present disclosure is also directed to stirred yogurts produced by the methods discussed above, in one or more embodiments.

The fat and protein contents of the stirred yogurts produced by the methods herein are the same or substantially the same as the fat and protein contents of the raw milk utilized (described previously), since the fat and protein contents do not substantially change after lactic acid fermentation. While the stirred yogurt in the present disclosure is not particularly limited, low-fat stirred yogurt is preferable, for example stirred yogurt having a fat content of not more than 1.5 wt. %, based on a total weight of the stirred yogurt. Further, the stirred yogurt generally has a protein content of not more than 5.5 wt. %, preferably not more than 5 wt. %, preferably not more than 4.5 wt. %, preferably not more than 4.2 wt. %, based on a total weight of the stirred yogurt.

As the stirred yogurts of the present disclosure are made through the modification of proteins contained in raw milk with protein glutaminase, in which at least some of the glutamine residues are deamidated with the protein glutaminase enzyme, the stirred yogurts may contain fewer total glutamine residues than a total number of glutamine residues present in the raw milk used to make the stirred yogurt.

The stirred yogurts also comprise live Streptococcus thermophilus and Lactobacillus delbrueckii Subsp. Bulgaricus, and optionally other live microorganisms such as Lactobacillus delbrueckii Subsp. lactis, Bifidobacterium animalis Subsp. lactis, Lactococcus lactis, Lactobacillus acidophilus, and Lactobacillus paracasei, or any microorganism derived therefrom. In particular, Lactobacillus delbrueckii Subsp. bulgaricus and Streptococcus thermophilus which are cultured simultaneously may be found to be live in the stirred yogurt products in an amount of at least 1 million, preferably at least 5 million, preferably at least 10 million colony-forming units (CFU)/g.

In accordance with the present disclosure, the stirred yogurts produced by the methods herein have excellent rheological properties (e.g., high viscosity and torque) and agreeable organoleptic properties (e.g., smoothness, firmness, mild acidic taste, low levels of syneresis, etc.). These advantageous properties are achievable through modification of raw milk with protein glutaminase prior to fermentation, without the need for cross-linking enzymes or thickeners, and as such, preferred stirred yogurts are those which are substantially free of, preferably completely free of (0 wt. %), cross-linking enzymes/deactivated cross-linking enzymes (e.g., transglutaminase) and/or thickeners (e.g., starch).

The viscosity of the stirred yogurts may vary considerably depending on the yogurt base used; however, desirable stirred yogurts generally have a viscosity of at least 100,000 cP, preferably at least 110,000 cP, preferably at least 115,000 cP, preferably at least 120,000 cP, preferably at least 125,000 cP, preferably at least 130,000 cP, preferably at least 132,000 cP, preferably at least 134,000 cP, preferably at least 136,000 cP, preferably at least 138,000 cP, preferably at least 140,000 cP, and up to 250,000 cP, preferably up to 200,000 cP, preferably up to 180,000 cP, preferably up to 150,000 cP, preferably up to 148,000 cP, preferably up to 146,000 cP, preferably up to 145,000 cP, preferably up to 144,000 cP, preferably up to 142,000 cP, as measured with a Brookfield DV-I viscometer (Helipath A spindle; 4.0 rpm; 40° F.). Particularly preferred are those stirred yogurts having a spoonable viscosity of at least 138,000 cP, preferably at least 140,000 cP, preferably at least 142,000 cP, preferably at least 144,000 cP.

A related measurement to viscosity is torque percent, or the amount of torque resistance measured (in terms of %) by a rotating spindle immersed in a material. In preferred embodiments, the stirred yogurts have a torque (%) of at least 6.4, preferably at least 6.5, preferably at least 6.6, preferably at least 6.7, preferably at least 6.8, preferably at least 6.9, preferably at least 7.0, and up to 7.5, preferably up to 7.4, preferably up to 7.3, preferably up to 7.2.

Various additives may be optionally incorporated into the stirred yogurts to provide a variety of sweetened/flavored/textured stirred yogurt products. For example, the stirred yogurts of the present disclosure may be optionally formulated with one or more additives including, but are not limited to, added dairy raw materials (e.g., cream); sugar or sweetening agents (e.g., sucrose, maltitol, sorbitol, lactose); oil and fat; emulsifiers; flavorings including seasonings and spices; colorants; antioxidants; fruits (e.g., fruit pulp and fruit juice of strawberry); cereals; thickeners (e.g., starch); nutritional substances such as vitamins, minerals, and fiber (e.g., vitamin A, vitamin B, vitamin D, calcium, zinc, iron, folic acid, riboflavin, dextrin); animal and/or vegetable protein (e.g., soybean protein, wheat protein, etc.); and solid foods (e.g., chocolate).

The examples below are intended to further illustrate the stirred yogurts, their properties, and their methods of manufacture, and are not intended to limit the scope of the claims.

### EXAMPLES

Stirred yogurt samples were prepared according to the procedures described in the following examples, and tested according to one or more testing procedures described below.

The gelatin used is a 250 bloom beef gelatin. The yeast extract used is AROMILD U, a yeast extract containing 8 wt. % of natural glutathione available from Kohjin. ACTIVA SYG is a combination of protein glutaminase and transglutaminase enzymes designed for dairy applications, available from Ajinomoto Foods Europe. ACTIVA MP is a transglutaminase enzyme designed for dairy applications, available from Ajinomoto Foods Europe. ACTIVA YG is a transglutaminase preparation including yeast extract designed for dairy applications, available from Ajinomoto Health & Nutrition North America, Inc. “PG” is a protein glutaminase enzyme (deamidating activity 500 U/g), available from Amano Enzyme, Inc.

### Testing

Testing of the prepared stirred yogurt samples was performed after cold storage at 5° C. at the designated time interval (e.g., after 1 day, after 25 days, etc.).

### <Viscosity>

The viscosity was measured with a Brookfield DV-I viscometer according to the following parameters:
Spindle; Helipath A
Speed; 4.0 rpm
Temperature; 40° F.

The viscosity results are presented in terms of centipoise (cP).

### <Torque>

The torque was measured simultaneously with the viscosity using the viscosity procedure above (the viscosity and torque were both selected as automatic outputs as the Brookfield DV-I viscometer reads the sample). The torque results are presented in terms of percent (%).

### <Organoleptic Analysis>

The prepared stirred yogurt samples were evaluated by trained panelists with experience in profiling and prescreened for their sensory acuity. The trained panelists evaluated the prepared stirred yogurt samples according to one or more of appearance (e.g., amount of syneresis), texture, and flavor.

### Example 1 (Comparative)

### Procedure:

Raw milk was adjusted to a fat content of 1.5 wt. % and a protein content of 4.2 wt. %.
   Stabilizer was added at designated level.
   The raw milk was warmed at 50° C. for 30 minutes and then heat sterilized at 85° C. with stirring for 20 minutes.
   The raw milk was then cooled to 43° C.
The starter culture (YOFLEX PREMIUM 1.0 available from Chr. Hansen) was then added in an amount of 0.02 wt. %.
The protein modification enzyme was then added at the designated level and the raw milk was incubated (fermented) at 43° C. until a pH of 4.6 was reached.
The resulting yogurt was stirred.
   The stirred yogurt was stored at 5° C. for 7 days and then evaluated for viscosity, torque, and organoleptic properties.

### Results:

TABLE 1
Evaluation of Samples #1-2 after 7 days of cold storage

| | | Protein | | |
|---|---|---|---|---|
| | Stabilizer | modification | | |
| | Mixture of pectin and | enzyme | Viscosity | Torque |
| Sample # | modified food starch | ACTIVA MP | (cP) | (%) |
| 1 | 1 wt. % | — | 37,765 | 1.9 |
| 2 | 1 wt. % | 200 ppm | 49,231 | 2.5 |

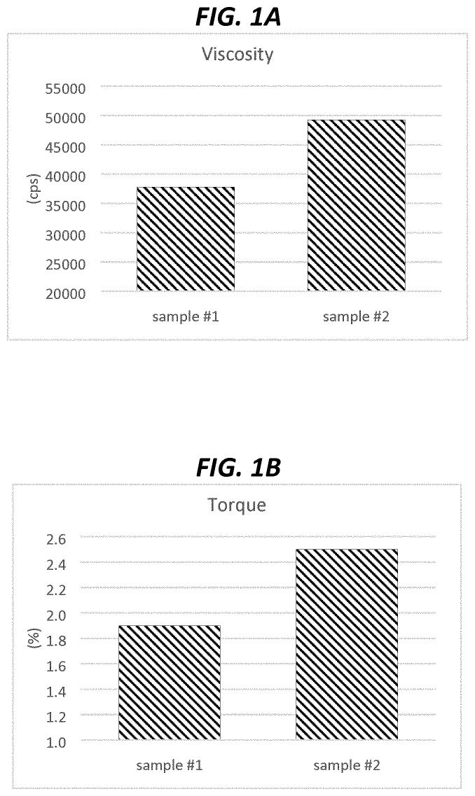

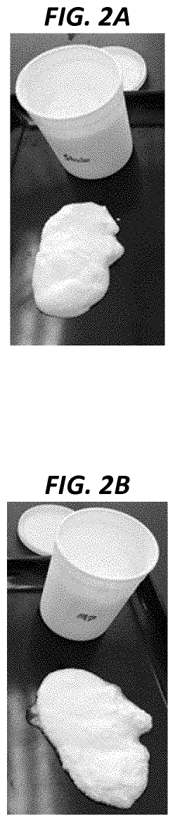

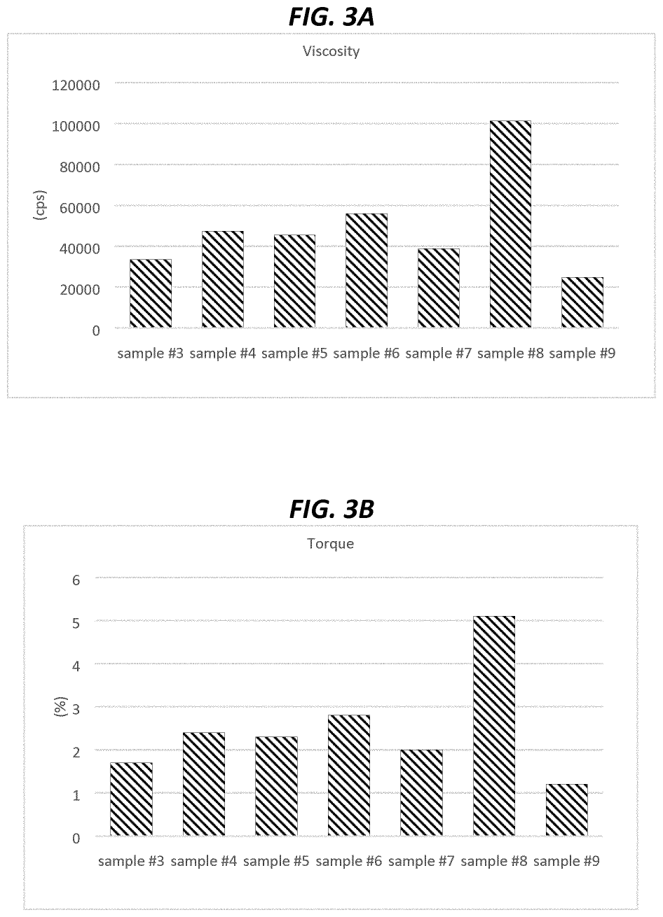

The addition of 200 ppm transglutaminase enzyme (ACTIVA MP) was found to provide a modest increase in viscosity and torque as can be seen in Table 1 and FIGS. 1A-1B. However, organoleptic analysis of sample #2 revealed an unsatisfactory lumpy appearance compared to sample #1 (control) (FIGS. 2A and 2B).

### Example 2 (Comparative)

Procedure:
Raw milk was adjusted to a fat content of 1.5 wt. % and a protein content of 4.2 wt. %.
   Stabilizer was added at designated level.
   The raw milk was warmed at 50° C. for 30 minutes and then heat sterilized at 85° C. with stirring for 20 minutes.
   The raw milk was then cooled to 43° C.
The starter culture (YOFLEX PREMIUM 1.0 available from Chr. Hansen) was then added in an amount of 0.02 wt. %.
The protein modification enzyme(s) was then added at the designated level and the raw milk was incubated (fermented) at 43° C. until a pH of 4.6 was reached.
The resulting yogurt was stirred.
   The stirred yogurt was stored at 5° C. for 10 days and then evaluated for viscosity, torque, and organoleptic properties.

### Results:

TABLE 2
Evaluation of Samples #3-9 after 10 days of cold storage

| | Stabilizer | | | | | | | |
|---|---|---|---|---|---|---|---|---|
| | Modified | | | Protein modification enzyme | | | | |
| | food | | | ACTIVA | ACTIVA | | Viscosity | Torque |
| Sample # | starch | Pectin | Gelatin | SYG | MP | PG | (cP) | (%) |
| 3 | — | — | — | — | — | — | 33,474 | 1.7 |
| (control) | | | | | | | | |
| 4 | 1.5 wt. % | — | — | — | — | — | 47,368 | 2.4 |
| 5 | — | 0.18 wt. % | — | — | — | — | 45,444 | 2.3 |
| 6 | — | — | 0.4 wt. % | — | — | — | 56,000 | 2.8 |
| 7 | — | — | — | 50 ppm | — | — | 38,889 | 2.0 |
| 8 | — | — | — | — | 100 ppm | — | 101,368 | 5.1 |
| 9 | — | — | — | — | — | 50 ppm | 24,774 | 1.2 |

The results from enzymatic protein modification performed during fermentation can be seen from Table 2 and FIGS. 3A-3B. All stirred yogurt samples were found to have a higher viscosity and torque compared to control (sample #3), with the notable exception of sample #9—the sample prepared by adding protein glutaminase at the same time as the starter culture (during fermentation). Sample #9 showed significantly reduced viscosity and torque compared to control sample #3 as well as the stabilized samples #4-6. Also, the addition of a combination of protein glutaminase and transglutaminase enzymes (ACTIVA SYG, sample #7) provided only marginal improvements over control, and substantially lower viscosity/torque compared to stirred yogurts modified with transglutaminase enzyme alone (sample #8). These results indicate that the use of protein glutaminase during fermentation negatively affects the rheological properties of stirred yogurt.

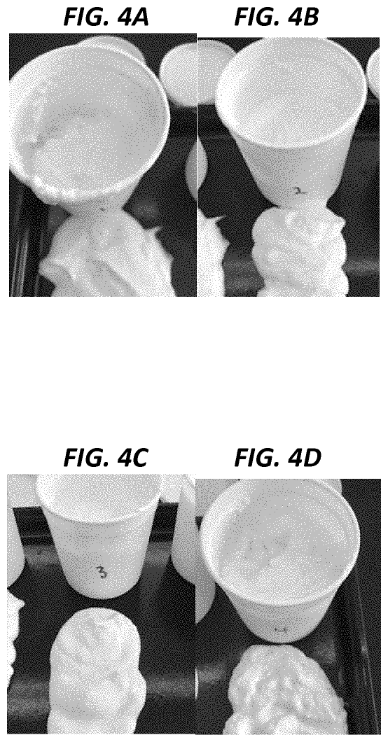

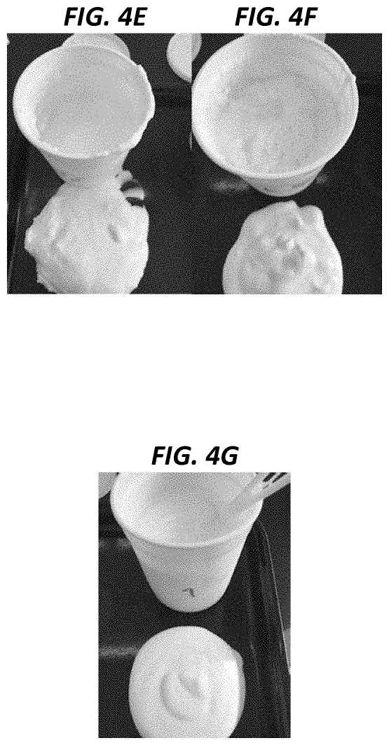

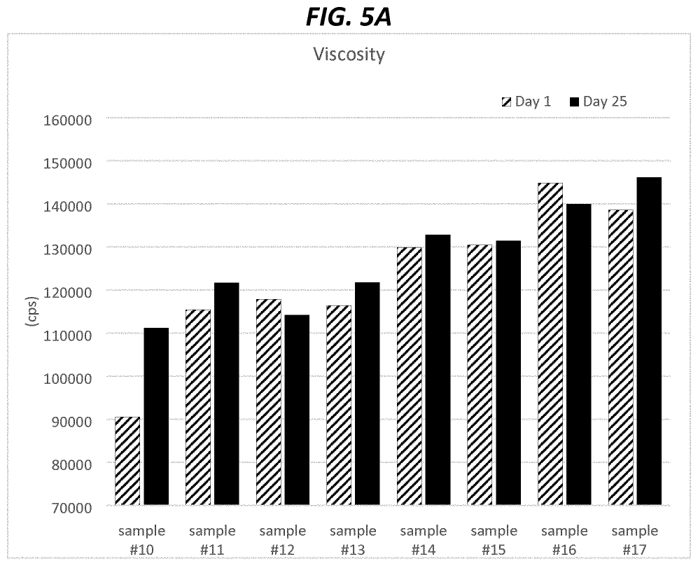

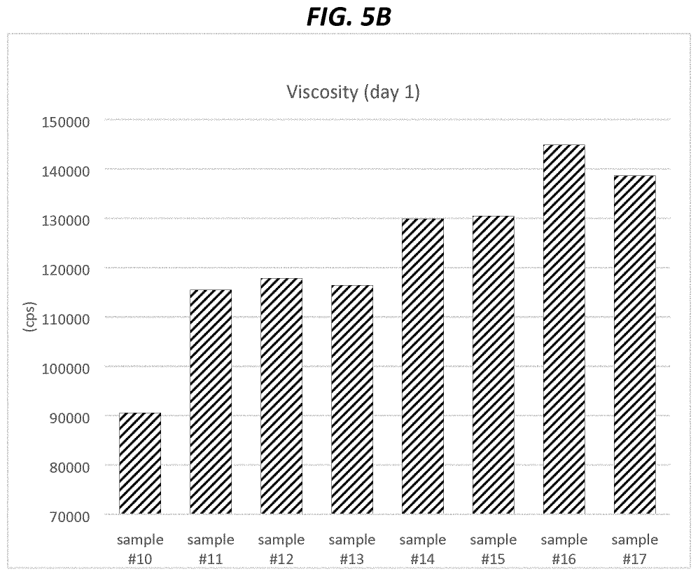

In terms of organoleptic analysis (FIGS. 4A-4G), sample #3 (control)(FIG. 4A) was found to have the most syneresis, followed by sample #7 (prepared with ACTIVA SYG)(FIG. 4E). The stirred yogurts with the thinnest mouthfeel were sample #3 (control)(FIG. 4A) and sample #9 (prepared with PG)(FIG. 4G) in accord with the viscosity findings, however, sample #9 was found to have a smoother texture than control.

### Example 3 (Inventive)

### Procedure:

Raw milk was adjusted to a fat content of 1.5 wt. % and a protein content of 4.2 wt. %.
   Stabilizer was added at designated level.
   The raw milk was warmed at 50° C. for 30 minutes.
The protein modification enzyme(s) and yeast extract were added at the designated levels.
   The temperature was held at 50° C. for the designated treatment time. For samples without protein modification enzymes, this treatment time was omitted.
   The samples were then heat sterilized at 85° C. with stirring for 20 minutes and then cooled to 43° C.
The starter culture (YOFLEX PREMIUM 1.0 available from Chr. Hansen) was then added in an amount of 0.02 wt. %, followed by incubation (fermentation) at 43° C. until a pH of 4.6 was reached.
The resulting yogurt was stirred.
   The stirred yogurt was stored at 5° C. and evaluated for viscosity, torque, and organoleptic properties after 1 day and 25 days of storage.

### Results:

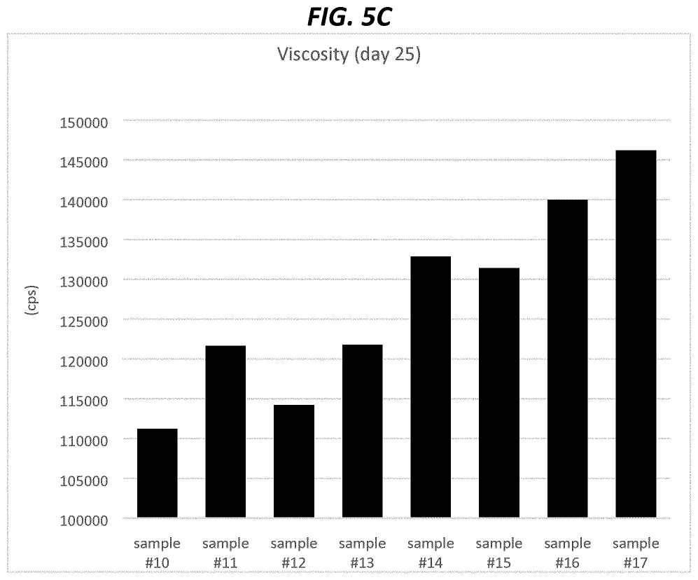

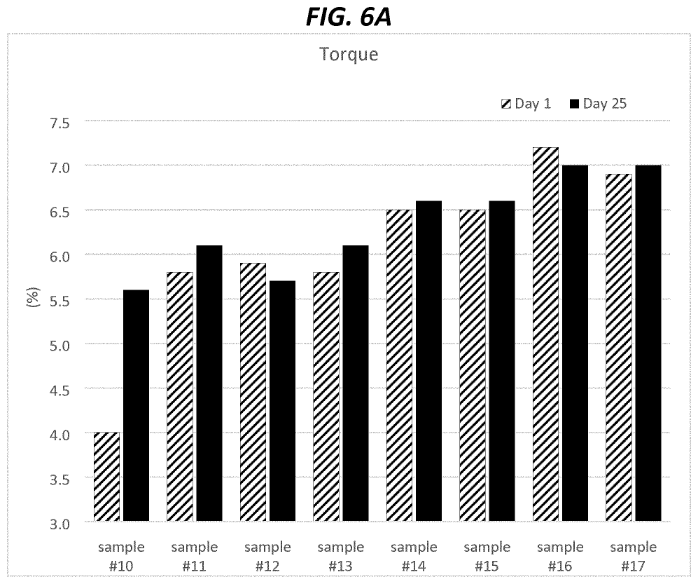

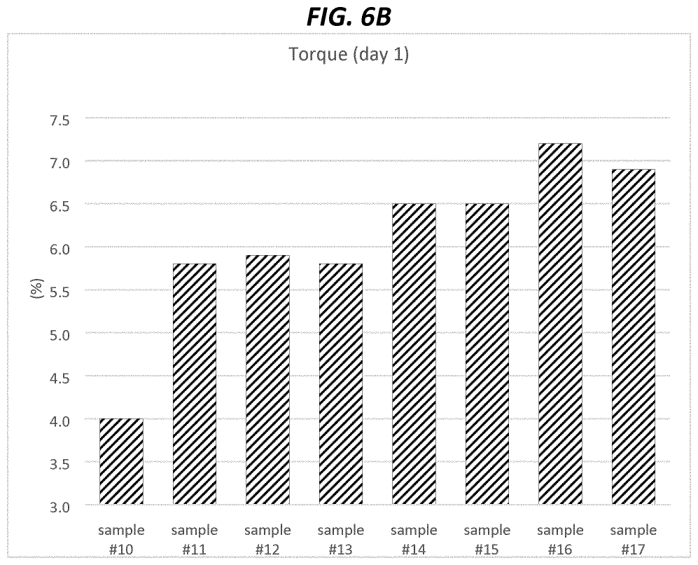

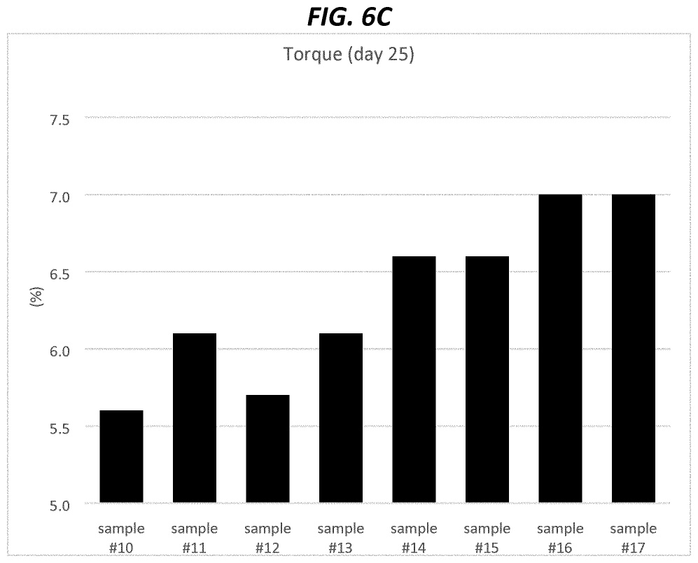

The results of enzymatic protein modification performed prior to fermentation are shown in Table 3 and FIGS. 5A-5C, 6A-6C. The addition of 200 ppm transglutaminase enzyme (ACTIVA YG) with a treatment time of 15 minutes (sample #12) gave the closest (lowest) viscosity to control (sample #10), with longer treatment times generally providing improved viscosities up until a treatment time of 45 minutes, after which only marginal improvements to day 1 viscosities were seen (samples #13-15).

Surprisingly, the best results were obtained by addition of protein glutaminase (sample #16) or protein glutaminase used in combination with transglutaminase (sample #17) prior to fermentation. In particular, pre-fermentation treatment using protein glutaminase as the only protein modification enzyme (sample #16) provided the highest day 1 viscosity of all samples tested, and an overall viscosity increase of 26 to 60% over the 25 day cold storage period compared to control. These results are in stark contrast to stirred yogurts produced by addition of protein glutaminase during fermentation (e.g., samples #7 and 9), where addition of protein glutaminase was found to negatively affect the rheological properties of stirred yogurt.

TABLE 3
Evaluation of Samples #10-17 after 1 and 25 days of cold storage

| | Stabilizer | | | | | | | | | |
|---|---|---|---|---|---|---|---|---|---|---|
| | Mixture of | Reducing | | | | | | | | |
| | pectin and | agent | Protein modification enzyme | | | | Viscosity | Viscosity | Torque | Torque |
| | modified | Yeast | ACTIVA | | ACTIVA | Treatment | (cP) | (cP) | (%) | (%) |
| Sample # | food starch | Extract | YG | PG | SYG | time | Day 1 | Day 25 | Day 1 | Day 25 |
| 10 | — | — | — | — | — | — | 90,533 | 111,200 | 4.0 | 5.6 |
| (control) | | | | | | | | | | |
| 11 | 1 wt. % | — | — | — | — | — | 115,451 | 121,647 | 5.8 | 6.1 |
| 12 | 1 wt. % | — | 200 ppm | — | — | 15 min | 117,833 | 114,222 | 5.9 | 5.7 |
| 13 | 1 wt. % | — | 200 ppm | — | — | 30 min | 116,364 | 121,778 | 5.8 | 6.1 |
| 14 | 1 wt. % | — | 200 ppm | — | — | 45 min | 129,877 | 132,848 | 6.5 | 6.6 |
| 15 | 1 wt. % | — | 200 ppm | — | — | 60 min | 130,440 | 131,419 | 6.5 | 6.6 |
| 16 | 1 wt. % | 100 ppm | — | 100 ppm | — | 60 min | 144,867 | 140,000 | 7.2 | 7.0 |
| 17 | 1 wt. % | 100 ppm | — | — | 100 ppm | 60 min | 138,632 | 146,187 | 6.9 | 7.0 |

TABLE 4
Organoleptic evaluation of samples #10-17 after 1 and 25 days of storage

| | Appearance | Texture | Flavor | |
|---|---|---|---|---|
| Sample # | Day 1 | Day 1 | Day 1 | Day 25 |
| 10 | Very slight free whey | Smooth, light | Clean, | No free whey, no other |
| (control) | on surface, shiny | body | mild acid | change |
| 11 | Slight free whey, slight | Smooth, more | Clean but | No free whey, no other |
| | grainy appearance, | body than #10 | muted | change |
| | more dull than #10 | | flavor | |
| 12 | Free whey similar to | Shorter texture | Similar to | No free whey, very |
| | #10 | than #10 | #10 | slight lumpy |
| | | | | appearance, no other |
| | | | | change |
| 13 | Similar to #10 | Similar to #12 | Similar to | No free whey, no other |
| | | | #10 | change |
| 14 | No free whey | Smooth body, | Similar to | No change |
| | | slightly more | #10 | |
| | | firm than #s 12 | | |
| | | and 13 | | |
| 15 | No free whey | Similar in body | Similar to | Very slight lumpy |
| | | to #14 | #10 | appearance |
| 16 | No free whey | Firmer, full body | Similar to | Very slight lumpy |
| | | | #10 | appearance, no other |
| | | | | change |
| 17 | No free whey | Similar to #16 | Similar to | No change |
| | | | #10 | |

In terms of organoleptic analysis (Table 4), sample #11 (prepared with only stabilizer) had the least favorable texture and flavor, with the modified food starch providing a muted flavor. The appearance of sample #11 was also dull and slightly grainy. Samples #16 and 17 had the most firm and full bodied texture and had the most pleasing organoleptic properties overall.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A method for producing a stirred yogurt, comprising:
treating raw milk with a protein glutaminase to form a modified milk;
fermenting the modified milk in the presence of a starter culture to form a yogurt; and
disrupting a gel structure of the yogurt to form the stirred yogurt.

2. The method of claim 1, wherein the raw milk has a fat content of up to 10 wt. %, based on a total weight of the raw milk.

3. The method of claim 1, wherein the raw milk has a protein content of 3 to 10 wt %, based on a total weight of the raw milk.

4. The method of claim 1, wherein the raw milk is treated with 10 to 500 ppm of the protein glutaminase, based on a total weight of the raw milk.

5. The method of claim 1, wherein the raw milk is treated with the protein glutaminase in an amount of 0.01 to 5 U per gram of protein in the raw milk.

6. The method of claim 1, wherein the raw milk is treated with the protein glutaminase at a treatment temperature of up to 50° C.

7. The method of claim 1, wherein the raw milk is treated with the protein glutaminase for a duration of 15 minutes to 24 hours.

8. The method of claim 1, further comprising heat sterilizing the modified milk after treating the raw milk with the protein glutaminase, and prior to fermenting the modified milk.

9. The method of claim 8, wherein the heat sterilizing is performed at a temperature 70 to 95° C. for 1 to 30 minutes.

10. The method of claim 1, further comprising adding a stabilizer to the raw milk prior to treating the raw milk with the protein glutaminase.

11. The method of claim 1, further comprising treating the raw milk with a reducing agent prior to, or concurrently with, the treating of the raw milk with the protein glutaminase.

12. The method of claim 11, wherein the raw milk is treated concurrently with the reducing agent and the protein glutaminase.

13. The method of claim 11, wherein the raw milk is treated with 10 to 500 ppm of the reducing agent, based on a total weight of the raw milk.

14. The method of claim 11, wherein the reducing agent is yeast extract.

15. The method of claim 1, wherein the modified milk is fermented with 0.0001 to 1 wt. % of the starter culture, based on a total weight of the modified milk.

16. The method of claim 1, which does not involve the use of a thickener.

17. The method of claim 1, further comprising treating the raw milk with a transglutaminase enzyme concurrently with the treating of the raw milk with the protein glutaminase.

18. The method of claim 1, wherein the protein glutaminase is the only enzyme utilized that acts on glutamine residues of milk proteins contained in the raw milk.

19. A stirred yogurt produced by the method of claim 1.
